(19) United States
(12) Patent Application Publication
(10) Pub. No.: US 2020/0029707 A1
(43) Pub. Date: Jan. 30, 2020

(54) WORKPLACE SYSTEM AND METHOD FOR CONTROLLING A WORKPLACE SYSTEM

(71) Applicant: LOGICDATA Electronic & Software Entwicklungs GmbH, DEUTSCHLANDSBERG (AT)

(72) Inventor: Stefan LUKAS, Preding (AT)

(21) Appl. No.: 16/499,193

(22) PCT Filed: Mar. 15, 2018
(86) PCT No.: PCT/EP2018/056563

§ 371 Date: Sep. 27, 2019

(30) Foreign Application Priority Data
Apr. 12, 2017 (DE) 10 2017 107 913.0

(51) Int. Cl. A47C 31/12; A61B 5/00; A47C 3/30

(52) U.S. Cl. CPC A47C 31/126; A61B 5/4561; A47B 2009/006; A47C 3/30; A61B 2503/24; A61B 5/486

(57) ABSTRACT

A workplace system comprises a work table, a height-adjustable work chair with a gas spring device, a plurality of sensors, an evaluation unit and a feedback device. At least one of the plurality of sensors is mounted on the worktable. At least one further of the plurality of sensors is mounted on or in the gas spring device and arranged to detect posture data of a user of the work chair. The evaluation unit is configured to receive the posture data and further measurement data from the plurality of sensors, to determine a well-being measure of the user on the basis of a joint evaluation of the received posture data and the measurement data, and to control the feedback device as a function of the well-being measure.

### BACKGROUND OF THE INVENTION

The present disclosure relates to a workplace system and a method for controlling a workplace system.

For users of workplaces, especially in the office sector, it is becoming increasingly important to ensure that the workplaces are used both ergonomically and efficiently. Modern office furniture offers multiple adjustment possibilities. For example, adjustable tables can be found where the height can be adjusted manually or by motors via the table frame. Office chairs respectively work chairs can also be adjusted in their position, especially height.

Current workplace systems already include sensors, e.g. force sensors in the table to detect a pinch, IR or ultrasonic sensors in the table to detect the presence of a user, or sensors in a seat of the work chair to detect a user posture.

### SUMMARY OF THE INVENTION

The present disclosure provides an efficient concept for the evaluation of sensor data in a workplace system that improves or facilitates the use of the workplace system.

The efficient concept is based on the idea that although a large amount of sensor data is available at the worktable, these sensor data regularly have too little informative value about the user's well-being. It has now been found that an essential element for such an assessment is the user's posture on the work chair. In particular, posture data acquired via a sensor on or in a gas spring device of the work chair make it possible to determine a meaningful measure of the user's well-being. This is done in particular by combining or jointly evaluating the posture data from the gas spring device and further measurement data from other sensors of the workplace system. Advantageously, at least measurement data from a sensor on the worktable are evaluated. Depending on the measure of well-being, which is determined in the joint evaluation, feedback to the user can be generated. Such a feedback can be a pure signaling, or also a change of a setting at the workplace system.

For example, an implementation form of a workplace system according to the efficient concept comprises a work table, a height-adjustable work chair with a gas spring device, a plurality of sensors, an evaluation unit and a feedback device. At least one of the plurality of sensors is mounted on the worktable. At least one further of the plurality of sensors is mounted on or in the gas spring device and is adapted to detect posture data of a user of the work chair. The evaluation unit is configured to receive the posture data and further measurement data from the plurality of sensors, to determine a well-being measure of the user on the basis of a joint evaluation of the received posture data and measurement data and to control the feedback device depending on the well-being measure.

Such a workplace system can be described as intelligent, as it uses sensor technology and pattern recognition to recognize the behavior and well-being of the user and triggers actions that are beneficial to the ergonomics, health and well-being of the user.

The workplace system thus represents a solution for the general trend of “beneficial intelligence”, which applied to the furniture industry means: furniture of a workplace system analyze the workplace and the user independently, and take actions to create the best possible working conditions and/or support the user in improving them.

The well-being measure represents, for example, a well-being of the user and/or a behavior of the user.

For example, the collected data is not simply presented to the user, but the user is playfully motivated to move around the workplace. For this purpose, the data of the available sensors can be used in the context of a game running on a mobile device of the user or on the PC.

Alternatively or additionally, the feedback device comprises at least one actuator which is also used for an adjustment function of the workplace system. It is also possible that the feedback device comprises at least one optical and/or tactile and/or haptic and/or acoustic signal transmitter.

In various implementations, the evaluation unit is configured to receive measurement data from a biometric sensor, which records biometric data of the user, and to determine the well-being measure additionally on the basis of the received measurement data of the biometric sensor. For example, the biometric sensor is comprised of a device worn by the user, especially on the body. Such a device is for example a fitness tracker, a chest strap, a wristband, a watch with sensors or the like. Such devices are also referred to by the English term “wearable”.

Furthermore, the evaluation unit may be configured to receive measurement data from a mobile communication device of the user and to determine the well-being measure additionally on the basis of the received measurement data of the mobile communication device.

The specific characteristic is to use the data from a biometric sensor, a communication device or other sensors, which are not contained in furniture components, in combination with data, which are recorded by sensors in a furniture component, to determine posture and well-being.

In addition, the measurement data used can also be supplied by sensors in a building technology system, for example in a so-called Smart Home or Smart Office. Examples include temperature sensors, presence detectors, brightness sensors, sensors for measuring air quality or the like.

In various implementations, the evaluation unit is configured, during the joint evaluation, to evaluate a temporal course of the received posture data and measurement data in order to determine at least one pattern and to determine the well-being measure on the basis of the at least one pattern.

For example, regularities, repetitions, similarities or laws can be identified from the course of the sensor data over time. These patterns allow the evaluation unit to draw conclusions about the environmental conditions mentioned and the user and to take appropriate actions. In the simplest case, the actions are a simple feedback to the user. In more complex scenarios, workplace system settings such as table height, lighting setting, temperature setting, etc. can also be changed.

Generally speaking, other, new information can be derived by combining and taking into account the temporal course.

For example, the well-being measure and/or pattern contains change information associated with a change in the user's sitting position and/or a change in the user's position relative to the worktable. The feedback device comprises at least one actuator, which is used, for example, for height adjustment of the worktable. The evaluation unit is set up to control the feedback device for height adjustment of the worktable on the basis of the change information. Alternatively or additionally, other actions can be initiated on the basis of the change information.

The following scenarios can be thought of as examples: Rising from the chair and manual height adjustment of the table; rising from the chair and automatic height adjustment of the table; leaving the workplace and automatic height adjustment of the table; sitting down and automatic height adjustment of the table. These scenarios will be explained later in more detail in connection with the figures.

The use of a sensor in or on a gas spring makes it possible to provide a wide variety of significant information with little measuring effort. For example, the posture data comprises at least one of the following: a user's center of gravity on the work chair; a user's inclination on the work chair; a user's sitting angle on the work chair; the user's weight; a height position of the work chair; a rotation angle of the work chair.

In combination with the other measurement data, the posture data mentioned allow far-reaching conclusions to be drawn about the user's well-being, working position and/or working condition.

In various implementations, the workplace system further comprises a data processing device, e.g. a workplace computer, a tablet computer or a mobile telephone, wherein the evaluation unit comprises the data processing device or is formed by the data processing device.

A peculiarity here is that the combined evaluation of the sensor data of individual sensors is not carried out at the location of the measurement but in a PC/Smartphone and that the computing power provided by these devices is used, whereby for instance the energy consumption in the furniture components, e.g. “intelligent” furniture components, can be kept low.

The sensors and processors used in intelligent “things”, e.g. intelligent furniture systems, generally have to be small and energy-saving. The computing power required for the combination of sensors and subsequent evaluation is therefore usually not possible there. Instead of a separate evaluation unit, it is therefore proposed to perform the evaluation using typical devices available at a workplace, e.g. workplace computers, tablet computers, mobile phones or other mobile devices.

In various implementations, the evaluation unit is configured to repeatedly determine the level of sensitivity and to control the feedback device as a function of the level of sensitivity determined in each case. In the process, a difference measure is determined between the determined well-being measures. After a certain repetition, the feedback device is additionally controlled depending on whether the difference measure exceeds or is below a threshold value.

According to the efficient concept, a method for controlling a workplace system is also proposed. The workplace system comprises, accordingly, a work table, a height-adjustable work chair having a gas spring device, and a plurality of sensors, wherein at least one of the plurality of sensors is mounted on the work table and at least one further of the plurality of sensors is mounted on or in the gas spring device and is adapted to detect posture data of a user of the work chair. According to the method, the posture data and further measurement data are received by the plurality of sensors. The well-being measure of the user is determined on the basis of a joint evaluation of the received posture data and measurement data, and at least one component of the workplace system is controlled as a function of the well-being measure in order to carry out a feedback.

Developments of the method result for the skilled person directly from the different implementations for the workplace system described above.

### BRIEF DESCRIPTION OF THE DRAWINGS

The invention will be explained in more detail in the following by means of examples with reference to the drawings. Here similar elements or elements of similar functions are designated with the same reference signs. Therefore, a repeated explanation of individual elements may be omitted.

In the drawings:

FIG. 1 shows an example of a workplace system;

FIG. 2 shows an example of a work chair;

FIG. 3 shows an exemplary flow chart for a method for controlling a workplace system;

FIG. 4 shows another exemplary flowchart for a method of controlling a workplace system; and

FIGS. 5A and 5B shows exemplary illustrations for the application of a method for the control of a workplace system.

### DETAILED DESCRIPTION

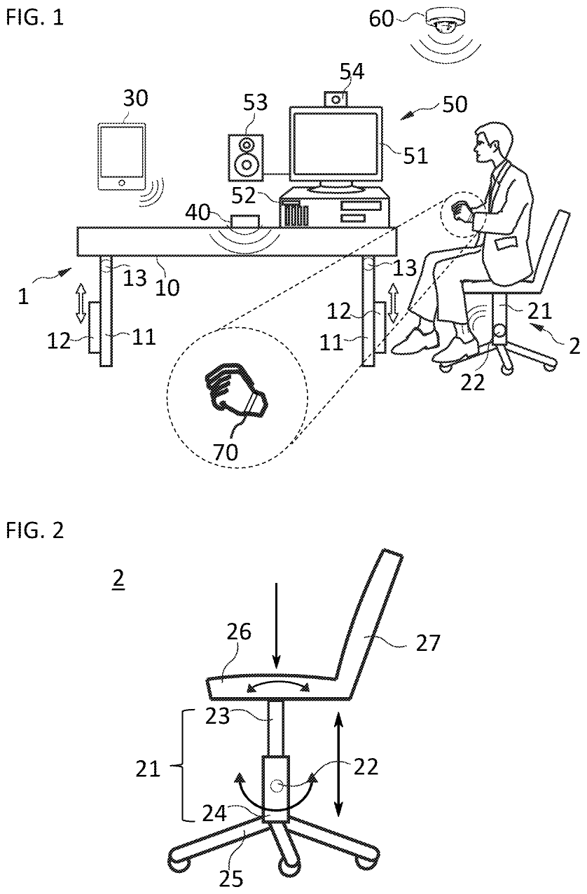

FIG. 1 shows an example of a workplace system where the efficient concept can be applied. A large number of components and/or sensors that can be used are shown as examples. In a practical implementation, however, it is not essential that all components and/or sensors are present.

The workplace system comprises a worktable 1 with a tabletop 10 mounted on a height-adjustable frame 11. Adjustment is made, for example, via corresponding actuators 12. For example, one or more force sensors 13 are mounted in the frame 11 or in/on the table top 10, which can be used to measure a force and/or deformation. For example, such sensors are used for collision detection.

The workplace system also includes a work chair 2 equipped with a gas spring device 21. A sensor 22 is mounted on or in the gas spring device 21 and can be used to measure at least one parameter of the gas spring device 21. For example, the sensor provides 22 posture data of the user, who is represented as sitting on the work chair 2.

The workplace system is also equipped with a tablet computer 30 as an example of a mobile communication device, which is used, for example, as an evaluation unit according to the efficient concept. Furthermore, a feedback device 40 is symbolically represented, which is formed, for example, by a tactile or haptic signal transmitter. Furthermore, a workplace computer system 50 with a screen 51, a computer housing 52, a loudspeaker 53 and a camera 54 is shown. For example, loudspeaker 53 can be used as an acoustic signal generator. The camera 54, for example, can be used as a supplier of measurement data. Screen 51 can be used to display visual feedback from the workplace system in addition to standard computer applications. As an alternative to the tablet computer 30, the workplace computer system 50 can also be used as an evaluation unit.

For example, a presence detector 60 is also provided in the workplace system, which can detect the presence of the user.

The user, for example, wears a sensor wristband 70 in the illustration, which is shown in magnification. This wristband 70 features one or more biometric sensors that can measure and record various body functions of the user. Alternatively, the wristband 70 can also be designed as a so-called Smartwatch, which contains corresponding sensors.

FIG. 2 shows in detail a possible design of the work chair 2. The work chair 2 comprises as an essential element the gas spring device 21 comprising a piston 23 which can move into a cylinder 24. For example, sensor 22 is mounted on cylinder 24 or in cylinder 24. The cylinder 24 is connected to a base 25, which forms the supporting surface of the work chair 2. A seat 26 with backrest 27 is mounted on the piston 23.

The arrows depicted in FIG. 2 indicate various directions of movement and forces which the work chair can perform or which have an effect on it. One or more of these directions of movement or the resulting positions can be detected via sensor 22. For example, a rotation angle and/or a height position of the work chair 2 can be detected. Furthermore, it is generally possible to determine a tilting of the seat surface 26. All degrees of freedom can be taken into account, whereas in the present diagram only one tilting direction is indicated. Furthermore, a force on the seat surface 26, for example a weight force of the user, can be measured.

The data collected by the sensor 22 can be understood as posture data, in particular of the user on the work chair 2. The posture data comprise, for example, at least one of the following: a center of gravity of the user on the work chair; an inclination of the user on the work chair; a sitting angle of the user on the work chair; the weight of the user; a height position of the work chair; a rotation angle of the work chair.

A detailed configuration of a gas spring device can be found, for example, in the German patent application number 102016102891.6 of the applicant and in the international patent applications PCT/EP2017/053706 and PCT/EP2017/053708, the contents of which are fully incorporated in their entirety by reference insofar as the law permits.

With the various embodiments of the workplace system and the method for controlling a workplace system, in the simplest case sensors are used in the table and in the gas spring device in the workchair in order to be able to deduce the behavior and well-being of a user by combination, i.e. joint evaluation. Further, non-exclusive examples of sensor components of an intelligent workplace system are listed below:
Table with presence sensor(s) (IR, ultrasound),
Table with force sensor(s)
Work chair with sensors in backrest, base, seat, armrests (“intelligent chair”)
“Wearable” with sensors for biometric data acquisition (heartbeat, heartbeat variability HRV, blood pressure, body temperature)
“Wearable” with gyro sensor (e.g. to detect a certain angular position of a person while sitting)
Chest straps for heartbeat measurement
Mobile devices, e.g. Smartphone or Tablet as sensor for the activity of a user (e.g. pedometer)
Workplace PC or its keyboard, mouse as sensor for the presence of a user
Doormats under the table or the chair
Cameras (face recognition, user identification, eye height-level recognition)
Building services as sensors (motion detectors)
time measurement

An intelligent workplace system is not limited to the combination of work chair and table, but can consist of any combination of the aforementioned sensors and actuators.

The well-being of a user at an office workplace is determined, among other things, by the possibility of being able to work in a concentrated manner. Concentration is influenced by temperature, noise level, air quality, light and other factors. Data from sensors that measure these influencing factors can therefore be advantageously combined to take measures that allow the user to concentrate more easily. The system can provide the user with feedback on factors that can have a negative effect on concentration.

The information derived from the evaluation unit by combining the sensor data can trigger various actions. Non-exclusive examples of this are:
Feedback via mobile device (acoustic, optical, text message, tactile by vibration)
Feedback via movement of a furniture component of the workplace system (e.g. vibration or height adjustment of an chair, table, etc.)
Feedback via acoustic, optical components attached to one of the furniture components (e.g. LEDs)
Feedback about optical components that are in the vicinity of the workplace (“Do not disturb sign” on the doorsign)
Control of building services (e.g. air conditioning, fans, blinds) to adapt the workplace climate
Controlling the table to adjust the height of the table top
Control of the chair to adapt massage, heating/cooling, height or hardness to the seat surface
Adjusting a monitor in height and eye distance/angle (adjustable monitor foot)
Controlling an aroma generator
Selecting a light (e.g. under the table top, table foot, . . . ) to make the user's condition visible to others (a kind of “do not disturb” display)

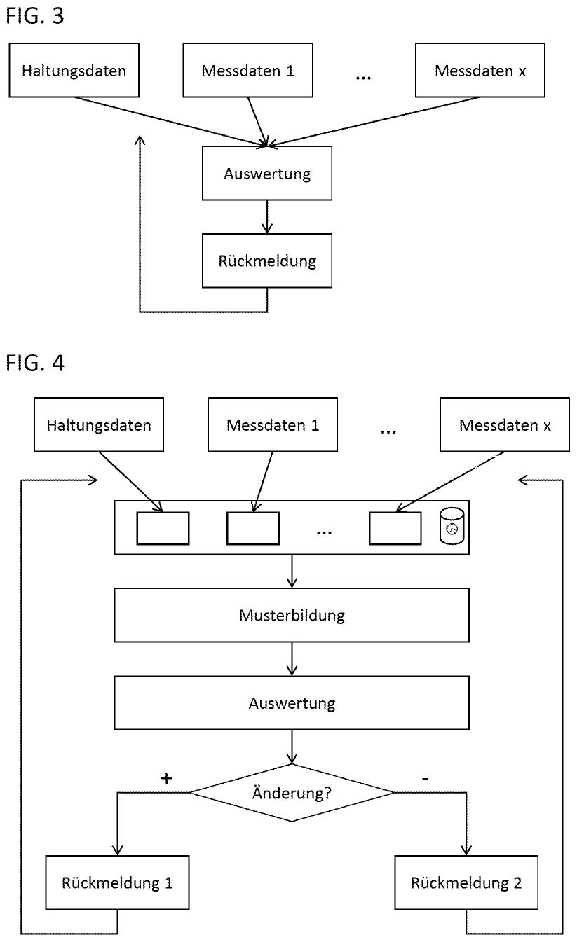

FIG. 3 is used as an example to describe the efficient concept with which posture data supplied by work chair 2 and measurement data from a wide variety of sources are jointly evaluated. The measurement data of at least one source preferably come from a sensor mounted on worktable 1.

In the evaluation, which is preferably carried out in the evaluation unit, a well-being measure of the user is determined on the basis of a joint evaluation of the received posture data and measurement data. This well-being measure serves as the basis for a feedback, for example via one of the aforementioned feedback devices. The acquisition of posture data or measurement data and their corresponding evaluation with feedback can take place iteratively, so that a continuous process is generally possible.

In a further development, also temporal courses of the posture data and/or measurement data are recorded and evaluated. This is shown in the exemplary flow chart in FIG. 4. The posture data or measurement data are stored in a buffer memory, for example, which enables access to a temporal course of the stored data as a result. From the stored data patterns can be formed, in particular by joint consideration of the stored data or the relationship between the stored data. However, the patterns can also indicate a significant change in posture data or measurement data at a certain point in time.

The well-being measure can be determined for example by evaluation or use of the determined patterns in order to form a basis for feedback via the workplace system.

For example, corresponding evaluation software in the evaluation unit uses the data from at least two different sensors of the at least two different components, determines patterns from the temporal course of the data and determines a feedback for the user from the patterns. Patterns are used e.g. for recognizing the person's well-being, for example in the form of fatigue, stress, excitement (emotions in general), pain, restless sitting behavior as a sign that the user does not feel comfortable, recognition of feelings via camera, breathing, concentration or the like.

Alternatively or additionally, the behavior of the person can be identified, e.g. in the form of getting up and leaving the workplace, getting up and standing still at the workplace, sitting down (e.g. by analyzing the progression of the weight force on the armchair), poor sitting posture, or the worsening of the sitting posture, incorrect eye distance/eye height in relation to a monitor, or the worsening of the same.

In the specific implementation shown in FIG. 4, a generally optional step is used to check whether there has been a change in the well-being measure compared with a previous evaluation in order to select the type of feedback depending on this. The well-being measure is thus determined repeatedly, whereby when testing for a change, a difference measure between the determined well-being measures is determined.

For example, the type of feedback depends on whether or not the difference measure exceeds or is below a previously set threshold value. This can also be made dependent on the number of completed feedback messages. This allows the implementation of certain feedback strategies with changing feedbacks, for example. The check of the changes implements, so to speak, a decision algorithm.

The advantage of using more than one sensor is that by combining the sensor data of several different sensors, a more reliable recognition of the well-being/behavior of a person is possible. If the data can still be interpreted in many ways on the basis of a single sensor, the result becomes more accurate and reliable by using several different sensors and such a decision algorithm. This decision algorithm can also include interaction with the person. The workplace system provides the person with feedback several times and, if, for example, the measured situation does not change, chooses a different strategy/action after the second feedback.

As an example, a force sensor can tell whether someone is standing up or sitting down. But only with a second sensor, e.g. a motion sensor or a gesture control via a mobile device, is it possible to distinguish whether the person stands up and leaves the workplace, or stands up to continue working while standing. If the workplace system detects the latter situation, an automatic adjustment of the table height can be carried out as an action, while an adjustment of the table height is unsuitable in the other situation.

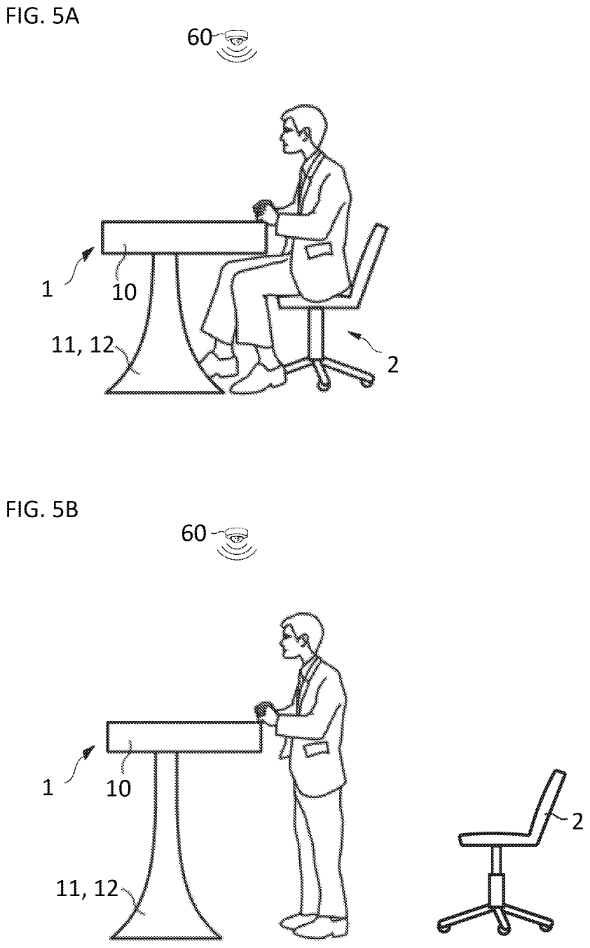

FIGS. 5A and 5B show exemplary illustrations for the application of a method for controlling a workplace system. FIG. 5A shows the user in a sitting position on work chair 2 at work table 1. In FIG. 5B, on the other hand, the user stands at work table 1, work chair 2 stands unused and unloaded next to or behind it. This illustrates two typical situations for using the workplace system.

On the basis of these illustrations different scenarios for the use of the workplace system will be explained.

A first scenario involves standing up and manually adjusting the height of the worktable. The aim here is that when the user stands up and pulls or pushes on the tabletop 10 (intuitive operation), the table should move up or down accordingly.

With the help of the intelligent workplace system and its sensors, the recognition of whether height adjustment is desired is now to be activated by standing up itself. For example, the following steps are performed:

The user stands up. The sensors of the gas spring device in work chair 2 detect that the user has got up. The evaluation unit now activates a mechanism that allows the height of the table top 10 to be adjusted by pulling or pushing. The signals of one or more force sensors, which are connected to the table top 10, are mainly used for the detection of pulling and pushing. For example, pulling causes an upward movement, while pushing causes a downward movement.

Adjustment by pulling/pushing is now activated for a certain period of time. If the user does not pull/push within this time, the mechanism is deactivated again. This means that after this time has elapsed, for example, the pulling/pushing no longer triggers any adjustment of the table top 10.

If the user pulls/pushes within this time period, he triggers the height adjustment (push down, pull up). The table moves as long as it is pulled/pushed. If the user interrupts pulling/pushing, the mechanism is deactivated after another short period of time. If the user pulls/pushes again before this short period of time has elapsed, the corresponding height adjustment is triggered again.

A second scenario concerns getting up and automatically adjusting the height of the worktable and is a modification of the first scenario. The aim is that when the user stands up, the table should automatically adjust to a predefined standing position.

This is conceived as an alternative to manual height adjustment and can be selected by the user, for instance. For example, the following steps are performed:

The user stands up. The sensors of the gas spring device in work chair 2 detect that the user has got up. If the evaluation unit detects from the presence sensor 60 signals that the user is not moving away from the table during the time period, it activates a mechanism after the time period has elapsed that moves the table top to the predefined standing position. While driving, for example, a force sensor is used for collision detection.

A third scenario concerns sitting down and automatic height adjustment. The aim is that when the user sits down, the table should automatically adjust to a predefined sitting position. Analogous to the second scenario, this is an alternative to the manual height adjustment according to the first scenario. For example, the following steps are performed:

The user sits down. The sensors of the gas spring device in the work chair detect sitting down. The evaluation unit activates a mechanism that brings the table top to the predefined sitting position. While driving, for example, a force sensor is used for collision detection.

A fourth scenario concerns getting up, walking away and automatic height adjustment. The aim is that when the user leaves the workplace, the table should move to a predefined rest position. For example, the following steps are performed:

The user stands or sits. The user leaves the workplace. A presence sensor 60 of the intelligent workplace system informs the evaluation unit that the user has left the workplace. Alternatively or additionally, a sensor worn by the user, e.g. a biometric sensor, can report the same information. The evaluation unit activates a mechanism that brings the table top to the predefined rest position. While driving, a force sensor can be used for collision detection.

In the following, further examples will be explained how the information from different measurement data or posture data can be used in the workplace system.

A combination of a center of gravity sensor and a force sensor in the back and/or armrest, for example, allows to determine various lying or semi-lying positions of the person and, in combination with the time the person is in this sitting position, to initiate actions to motivate the person to a better/healthier posture.

Frequent movement of a chair in combination with biometric data, e.g. breathing rate, pulse rate or the like, can indicate stress or inner restlessness.

Slight movements of a chair in combination with a high activity of the mouse can in turn indicate concentrated work.

Fatigue associated with a too high temperature can be used as a signal that cooling measures are being taken. A temperature measurement alone would not be meaningful enough, because the personal perception of temperature is different.

A measurement of the sitting angle in combination with a measurement of the center of gravity can be used, for example, to detect a sitting posture that is bent too far forward. The system could then propose a higher table position as feedback. If, however, the measured seat height matches the measured table height and it can be seen that the table height is already within the ergonomically correct range, then a suggestion for monitor height adjustment is appropriate. This can be done manually or by motor. Alternatively, a distance measurement can be performed to determine the distance between the monitor and the user's head or eyes.

If the center of gravity measurement measures a sitting position at the front edge of the seat surface and a force sensor located in the table measures a force caused by the supporting of the person (e.g. the person is leaning with his elbows), a proposal for height adjustment of the table can be made after a certain period of time in this position, for example.

It is known from flow theory that feeling a flow state is accompanied by a change in heart rate, heart rate variability (HRV) and skin conductance. By appropriate measurement and evaluation of the biometric data of a person, the flow experience can be trained and the advantages (satisfaction, productivity) associated with the flow condition can be achieved.

Sensors, for example, can use both unidirectional and bidirectional communication. Bidirectional communication allows the system to request data from sensors only when they are needed for decision making. Communication rates can be reduced, for example, by hiding unneeded sensors.

For example, brightness sensors of several intelligent workplaces in an office can be used to control the lighting in such a way that ideal brightness is available per workplace. For instance, if workplaces are not occupied (which can be detected by combining one or more presence sensors), individual office lamps, blinds or other dimming or lighting equipment controlled via building services can be deactivated to save energy.

In addition to the evaluation of the temporal course of sensor data, for example also the time instant is used in the evaluation. It is generally known that a person's performance is not always the same throughout the day, but is subject to periodic fluctuations. The time during which work was concentrated, measured by an intelligent workplace system using a combination of various sensors, can be supplied to the user as feedback. In this way one can get to know the personal performance curve.

The feedback to the user can be made dependent on this personal performance curve. For example, in the typical midday low, a feedback to a height adjustment (working in a standing position) or a request for fitness exercise may be more likely than in a high performance.

The data supplied by the sensors can also be used to “playfully” induce the user to be fit at the workplace (“Gamification of health and wellness”). Sensors of the intelligent workplace are used in a game program (e.g. on a PC or mobile device). This allows the combination of pauses or exercises necessary for health at the workplace with a playful incentive (e.g. collecting points, team-based, virtual character, which is further developed by the exercises). Among other things, the data from the sensors of the furniture components are used, for example, to record the execution of the exercises.

The number of available or used sensors/actuators can, for example, be configured by the user and thus adapted to the available sensors of the workplace system. Depending on the configured sensors, the system selects, for example, from the set of all algorithms those that can derive meaningful statements with these sensors.

For example, the algorithms can be adapted to the user. The adjustment is made either manually by the user by adjusting certain parameters of the patterns (sensitivity, response, time span, type of feedback/actuator selection giving feedback). However, it can be partially or completely supported by an automatic learning process. Settings can also be saved and recalled personalized.

1. A workplace system having a work table, a height-adjustable work chair with a gas spring device, a multiplicity of sensors, an evaluation unit and a feedback device, wherein
at least one of the plurality of sensors is mounted on the worktable;
at least one further of the plurality of sensors is mounted on or in the gas spring device and is adapted to detect posture data of a user of the work chair;
the evaluation unit is arranged to receive the posture data and further measurement data from the plurality of sensors, to determine a well-being measure of the user on the basis of a joint evaluation of the received posture data and the measurement data, and to control the feedback device as a function of the well-being measure.

2. The workplace system according to claim 1, wherein the evaluation unit is configured to receive measurement data from a biometric sensor that captures biometric data of the user and to determine the well-being measure additionally on the basis of the received measurement data of the biometric sensor.

3. The workplace system according to claim 2, wherein the biometric sensor is comprised of a device worn by the user, in particular worn on the body.

4. The workplace system according to claim 1, wherein the evaluation unit is configured to receive measurement data from a mobile communication device of the user and to determine the well-being measure additionally on the basis of the received measurement data of the mobile communication device.

5. The workplace system according to claim 1, wherein the evaluation unit is arranged to evaluate a temporal course of the received posture data and measurement data during the joint evaluation in order to determine at least one pattern and to determine the well-being measure on the basis of the at least one pattern.

6. The workplace system according to claim 5, wherein
the well-being measure includes change information associated with a change of a sitting position of the user and/or a change of a position of the user in relation to the work table;
the feedback device comprises at least one actuator which is used for height adjustment of the working table; and
the evaluation unit is arranged to control the feedback device for height adjustment on the basis of the change information.

7. The workplace system according to claim 1, wherein the posture data comprise at least one of the following:
the user's center of gravity on the work chair;
an inclination of the user on the work chair;
a sitting angle of the user on the work chair;
the weight of the user;
a height position of the work chair;
an angle of rotation of the work chair.

8. The workplace system according to claim 1, further comprising a data processing device, in particular a workplace computer, a tablet computer or a mobile telephone, wherein the evaluation unit comprises the data processing device or is formed by the data processing device.

9. The workplace system according to claim 1, wherein the feedback device comprises at least one actuator which is also used for an adjustment function of the workplace system.

10. The workplace system according to claim 1, wherein the feedback device comprises at least one optical and/or tactile and/or haptic and/or acoustic signal transmitter.

11. The workplace system according to claim 1, wherein the well-being measure represents a well-being of the user and/or a behavior of the user.

12. The workplace system according to claim 1, wherein the evaluation unit is configured,
to determine the well-being measure repeatedly and to control the feedback device as a function of the well-being measure determined in each case;
to determine a difference measure between the determined well-being measures; and
to additionally control the feedback device after a certain repetition depending on whether the difference measure exceeds or is below a threshold value.

13. A method for controlling a workplace system comprising a work table, a height-adjustable work chair having a gas spring device, and a plurality of sensors, wherein at least one of the plurality of sensors is mounted on the work table and at least one further of the plurality of sensors is mounted on or in the gas spring device and is arranged to detect posture data of a user of the work chair, the method comprising:
Receiving posture data and further measurement data from the plurality of sensors;
Determining a well-being measure of the user on the basis of a joint evaluation of the received posture data and the measurement data; and
Controlling of at least one component of the workplace system as a function of the well-being measure for carrying out a feedback.

14. The method according to claim 13, wherein the further measurement data comprise biometric data of the user and/or measurement data from a mobile communication device of the user.

15. The method according to claim 13, wherein a temporal course of the received posture data and measurement data is evaluated in the joint evaluation in order to determine at least one pattern, wherein the well-being measure is determined on the basis of the at least one pattern.

16. The method according to claim 15, wherein
the well-being measure includes change information associated with a change of a sitting position of the user and/or a change of a position of the user in relation to the worktable; and
a height adjustment of the worktable is effected on the basis of the change information.

17. The method according to claim 13, wherein the posture data comprise at least one of the following:
the user's center of gravity on the work chair;
an inclination of the user on the work chair;
a sitting angle of the user on the work chair;
the weight of the user;
a height position of the work chair;
an angle of rotation of the work chair.

18. The method according to claim 13, wherein the at least one component comprises an optical and/or tactile and/or haptic and/or acoustic signal transmitter and/or an actuator which is also used for an adjustment function of the workplace system.
